(19) United States
(12) Patent Application Publication
(10) Pub. No.: US 2023/0389822 A1
(43) Pub. Date: Dec. 7, 2023

(54) DEVICES AND SYSTEMS FOR DETERMINING AND ADDRESSING PHYSIOLOGICAL IMPAIRMENTS

(71) Applicant: AT&T Intellectual Property I, L.P., Atlanta, GA (US)

(72) Inventors: Rashmi Palamadai, Naperville, IL (US); Brian Novack, St. Louis, MO (US); Eric Zavesky, Austin, TX (US)

(21) Appl. No.: 17/829,930

(22) Filed: Jun. 1, 2022

(51) Int. Cl. A61B 5/11; A61B 5/0205; A61B 5/083

(52) U.S. Cl. CPC A61B 5/1118; A61B 5/02055; A61B 5/0833; A61B 2503/10

(57) ABSTRACT

A processing system may obtain first biometric data of a user from at least one biometric device for a physical activity via at least a first exercise apparatus for a plurality of performance levels, where the obtaining includes associating biometric output levels from the first biometric data with corresponding performance levels of the plurality of performance levels as baseline biometric output levels. The processing system may next detect a performance of the physical activity by the user via the at least the first exercise apparatus, obtain a first performance level and second biometric data associated with the performance of the physical activity, the second biometric data indicating a first biometric output level, detect that the first biometric output level is elevated at the first performance level, and generate an instruction for the user to engage in a reduced performance level for the physical activity or a second physical activity.

The present disclosure relates generally to exercise apparatuses, biometric devices, and systems, and more particularly to methods, computer-readable media, and apparatuses for generating an instruction for a user to engage in a reduced performance level in response to detecting that a biometric output level of the user for a first physical activity via a first exercise apparatus is elevated as compared to a baseline biometric output level at a first performance level.

### BACKGROUND

There exist numerous different kinds of exercise equipment for users to engage in fitness and health activities. For instance, such equipment may include treadmills, stationary cycles, resistance exercise machines, free weights, elliptical machines, etc., as well as bicycles, boats/shells (e.g., for rowing), and so forth. In some cases, these exercise apparatuses may include network communication capabilities and may further include one or more sensors. For instance, a stationary cycle may include a screen for displaying online group exercise classes delivered via the Internet. In one example, a stationary cycle may also measure a speed at which a user is currently operating the device and may further report the speed to the user via the screen, as well as to a course instructor and/or to the entire class (e.g., to display on a “leaderboard” that may be presented to all users in the class). In addition, a stationary cycle may include a sensor that may measure heart rate/pulse via hand grips that may be engaged by a user. Similar features may also be provided by treadmills, for example, or other exercise equipment.

### SUMMARY

In one example, the present disclosure describes a method, computer-readable medium, and apparatus for generating an instruction for a user to engage in a reduced performance level in response to detecting that a biometric output level of the user for a first physical activity via a first exercise apparatus is elevated as compared to a baseline biometric output level at a first performance level. For instance, in one example, a processing system including at least one processor may obtain first biometric data of a user from at least one biometric device for a physical activity via at least a first exercise apparatus for a plurality of performance levels, where the obtaining includes associating biometric output levels from the first biometric data with corresponding performance levels of the plurality of performance levels as baseline biometric output levels. The processing system may next detect a performance of the physical activity by the user via the at least the first exercise apparatus, obtain a first performance level of the user and second biometric data of the user associated with the performance of the physical activity, where the second biometric data indicates a first biometric output level, and detect that the first biometric output level of the user is an elevated level for the user at the first performance level. The processing system may then generate an instruction for the user to engage in a reduced performance level for at least one of: the performance of the physical activity via the first exercise apparatus, a performance of at least a second physical activity via the first exercise apparatus, or the performance of the at least the second physical activity via a second exercise apparatus, where the reduced performance level is a lesser performance level of the plurality of performance levels as compared to the first performance level.

### BRIEF DESCRIPTION OF THE DRAWINGS

The teaching of the present disclosure can be readily understood by considering the following detailed description in conjunction with the accompanying drawings, in which:

FIG. 1 illustrates an example system related to the present disclosure;

FIG. 2 illustrates example tables of performance level records, biometric data records, baseline metrics, and peak performance levels, in accordance with the present disclosure;

FIG. 3 illustrates a flowchart of an example method for generating an instruction for a user to engage in a reduced performance level in response to detecting that a biometric output level of the user for a first physical activity via a first exercise apparatus is elevated as compared to a baseline biometric output level at a first performance level; and

FIG. 4 illustrates an example high-level block diagram of a computing device specifically programmed to perform the steps, functions, blocks, and/or operations described herein.

To facilitate understanding, identical reference numerals have been used, where possible, to designate identical elements that are common to the figures.

### DETAILED DESCRIPTION

Examples of the present disclosure describe methods, computer-readable media, and apparatuses for generating an instruction for a user to engage in a reduced performance level in response to detecting that a biometric output level of the user for a first physical activity via a first exercise apparatus is elevated as compared to a baseline biometric output level at a first performance level. In particular, examples of the present disclosure describe intelligent systems that include biometric sensor devices and exercise apparatuses that may provide directed guidance to a user in engaging in different physical activities (e.g., exercises) at different performance levels. In one example, a system of the present disclosure may obtain baseline biometric data of a user at different performance levels for one or more physical activities (e.g., obtaining first biometric data comprising a breathing rate, heart rate, etc. at a first speed on a treadmill, obtaining second biometric data comprising a breathing rate, heart rate, etc. at a second speed on the treadmill, and so forth). The system may then monitor the user in engaging in such physical activity, or activities, to detect a biometric output level of the user being elevated for a particular performance level as compared to the baseline. For instance, when such a condition is detected, the system may determine that the user is underperforming due to a physiological condition (e.g., and not a motivational problem). In addition, when such a condition is detected, the system may direct the user to engage in a reduced performance level (e.g., a lesser performance level as compared to the first performance level) via the same or different exercise apparatus and for a same or different physical activity. In particular, when a physiological condition is assumed to be an underlying issue, instead of a motivational problem, the system may attempt to reduce the performance level to avoid further injury, to keep the user at a target biometric output level, and so forth. On the other hand, if the user's biometric output level matches the baseline for a particular performance level, but the user has not reached a directed target performance level, the system may conclude that the lack of performance may be attributable to a motivational problem and may then provide a motivational message to the user.

In one example, the baseline biometric data may include range of motion data. In addition, the present disclosure may also incorporate personal medical records and factors like age, previous fitness level (e.g., a user ran a marathon last year and the biometric and performance levels were recorded at that time), current physical abilities (including accessibility-related aspects), previous injuries, and so forth. In accordance with the present disclosure, exercise apparatuses may include treadmills, rowing machines, stair-climber machines, elliptical machines, stationary cycles, resistance machines, weight machines, a pool with controllable water jets, and so forth. In one example, exercise apparatuses may also include smart shoes/sneakers, a bicycle, or other mobile exercise apparatuses equipped with network communication capabilities, as well as sensors for measuring performance levels (e.g., speed, acceleration, stride length, applied force, etc.). Similarly, biometric devices, or sensors, may include heart rate monitors, such as fitness bands, smartwatches with integrated sensors for heart rate, breathing rate, blood oxygen saturation, skin temperature, and other measurements, and so forth. In one example, an exercise apparatus may include one or more integrated biometric sensors, such as a heart rate monitor integrated into a stationary cycle that conducts measurements via handlebar grips, or the like.

In one example, the present disclosure may enable a user to select a goal/objective, e.g., to improve range, to heal, to provide physical therapy prior to or after a surgery, to regain a fitness level from five years ago, and so forth. In one example, the system may monitor in real time for a new injury that occurred during the session (or to detect a suspected injury that was not noted in a medical record or otherwise input to the system). In one example, the system may also generate an alert as to when a user should seek immediate medical attention, e.g., detecting when a user's has an extremely elevated heart rate or an irregular heart rate or pattern. For instance, the biometric data may indicate a fall or that the user has lost consciousness through sensors and alerts. For example, a treadmill may report that a user was previously jogging at 5 mph and that the treadmill no longer detects the user being on the treadmill (e.g., a lack of weight on the treadmill, etc.), while the treadmill was left running without being deactivated by the user. Thus, this data may corroborate the biometric data indicative of a fall and cause the system to generate an alert, e.g., to a caregiver or designated emergency contact, to emergency medical services, etc. In one example, the system may provide for a tiered, security/privacy-enabled communication. For instance, a doctor may have access to all data, while a sponsor may only access data that is relevant to an item being monitored.

In one example, the system may direct a user to engage in a physical activity at a particular performance level. For instance, the user may be directed to run at a particular speed on a treadmill, to engage in eight repetitions of bicep curls at a set weight, and so forth. Alternatively, or in addition, the user may be directed to engage in a physical activity at a particular biometric output level (e.g., to continue to run and maintain a heart rate of 160 beats per minute). In one example, the system may select one or more limitations to be implemented via an exercise apparatus, such as setting a maximum speed via a treadmill, setting a maximum resistance, setting an outer limit on a range of motion via a resistance machine, and so forth. In one example, the one or more limitations may be selected in response to an injury reported to the system by the user or via a digital medical record that is accessible to the system. Likewise, new physiological limitations (which may be caused by a new injury or other factors) may be detected as noted above, and the system may select and implement new limitations via one or more connected devices or services (e.g., reducing the maximum speed via a treadmill, reducing maximum resistance via a resistance machine, etc.).

In one example, the system may adjust a user-specific plan to account for a new injury and path to healing, and may tie-in to an original goal (or allow the user to set a new goal). For instance, a user may have set a goal of lifting 120 pounds for 10 repetitions by November 1. In addition, the system may have a physical activity schedule set for the user to assist the user in reaching such goal. However, when a new physiological limitation is detected, it may not be possible or safe, or the user may be cautioned that the goal should be adjusted. For instance, the user may be directed to engage in reduced resistance exercise(s) for at least two weeks, and then queried for any lingering pain or other physiological issue(s). The date for reaching the ultimate goal of lifting 120 pounds 10 times may be pushed back from November 1 to November 15, or subsequently if the user continues to confirm that the physiological issue(s) persist.

In an example in which the system is not integrated within a particular exercise apparatus, the system may push “safety” ranges to the equipment. In one example, if usage is beyond instructed limits or safety guidelines (e.g., determined by the system or by a prior user and/or medical professional specification), the system may escalate to involve a caregiver, to additionally query the user as to whether the user wishes to exceed the recommended range(s), to implement more frequent or accurate biometric monitoring, and so forth. In one example, the present disclosure may present an interactive dialog that may be used to confirm progress towards completing a specific physical activity, to provide for an interactive accept/reject sequence for the system to propose modifications, to obtain user affirmation, and so forth. In one example, the system may learn and/or react to the user based on the user's hesitation or inability to complete one or more directed actions. For instance, the system may detect that a user's biometric output level is elevated for a given performance level, and may thus determine that the user may be suffering from a potential physiological impairment. The system may then direct the user to engage in one or more physical activities at a reduced performance level (or at different reduced performance levels for different respective physical activities). However, the user may provide input to the system that the user cannot even perform at the reduced performance level(s). As such, the system may further reduce the directed performance level(s), or may direct the user to refrain from one or more physical activities entirely.

In one example, a user may first enroll in passive monitoring of activities (baseline actions) for the system to obtain baseline records. In one example, the system may also incorporate knowledge of the user's age, gender, and/or other physiological factors. In one example, the monitoring may serve as a pre-qualifier for participating in a particular activity. In one example, users may self-enroll one or more biometric devices/sensors for use, such as a heart rate monitor, a network of cameras to determine movement of limbs/body, etc. In one example, the system may locate available sensor devices or data sources by user opt-in. For instance, the system may pull range of motion data from a latest extended-reality exercise game the user is playing. Similarly, the system may incorporate visual samples, e.g., surface samples, infrared and thermal (subdermal) images, ultrasound measurements (e.g., for sensitivity), and so forth, depending upon the available biometric sensors.

In one example, the system may suggest that the user seek out specialized hardware to baseline the user's current physiological condition, e.g., to determine whether the user is injury free, or to determine that the user is affected by one or more preexisting injuries, which may be noted, such as muscle inflammation, bone damage, soft tissue damage, etc. In one example, the system may request the user to provide or perform “range of motion” comparison tests via an exercise apparatus, or may alternatively or additionally obtain the same or similar data from medical and/or physical therapy records—to assess capabilities with a current injury.

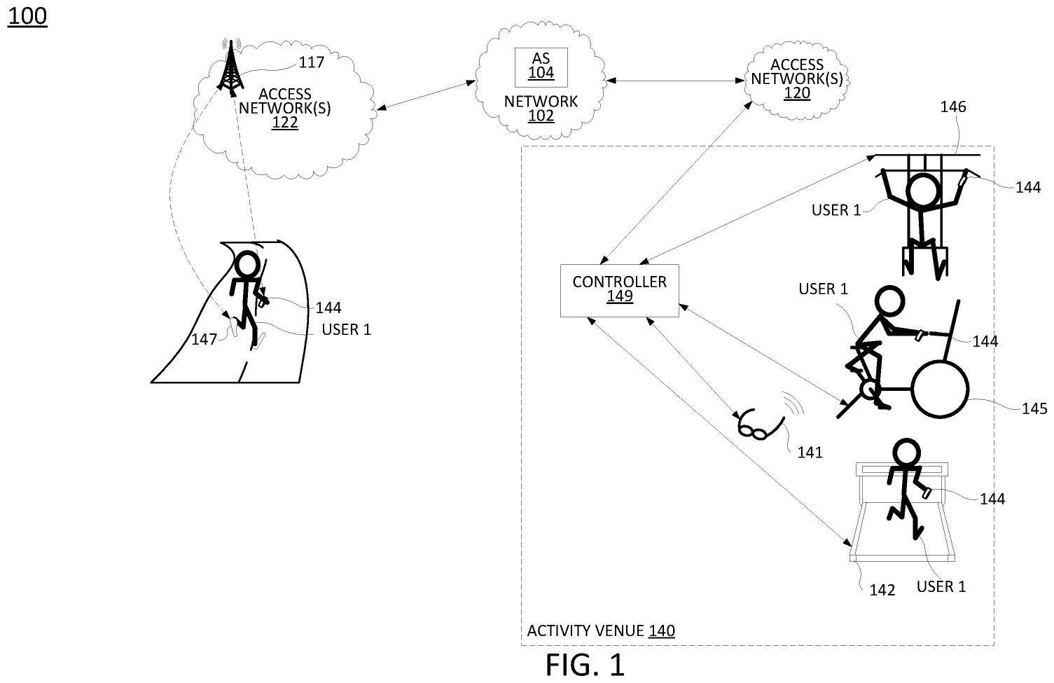

Notably, health/exercise equipment is increasingly democratized and self-service. However, those systems neither account for changes in behavior/ability due to injury (or detected anomalies), nor do they allow semi-automated adjustment of skill levels according to external changes. For example, existing systems may be designed by a professional and applied to the population in general. In contrast, the present examples detect baseline evolution via biometric sensors and exercise apparatuses, and continue to monitor a user through various physical activities via one or more exercise apparatuses. When a deviation in skill is detected, the entire digital profile of the user may be updated to accommodate new limitations and expectations for the user. In addition, in one example, instead of resetting a goal or changing resistance alone, those goals and limitations can be pushed from a user profile/agent to modify a plurality of physical activities that the user may engage in with the same or different exercise equipment (e.g. resistance, speed, time, distance, incline, etc.). In addition, in one example, changes and limitations that are being applied may be fully communicated to the user, thereby allowing the user to recalibrate and make the necessary adjustments. For instance, voice assistance and other agent-based communications or visualizations may be generated so that the user is able to understand the modified processes and so that the user is able to approve, reject, or modify such processes before being implemented by the system. These and other aspects of the present disclosure are discussed in greater detail below in connection with the examples of FIGS. 1-4.

To further aid in understanding the present disclosure, FIG. 1 illustrates an example system 100 in which examples of the present disclosure may operate. The system 100 may include any one or more types of communication networks, such as a traditional circuit switched network (e.g., a public switched telephone network (PSTN)) or a packet network such as an Internet Protocol (IP) network (e.g., an IP Multimedia Subsystem (IMS) network), an asynchronous transfer mode (ATM) network, a wireless network, a cellular network (e.g., in accordance with 3G, 4G/long term evolution (LTE), 5G, etc.), and the like related to the current disclosure. It should be noted that an IP network is broadly defined as a network that uses Internet Protocol to exchange data packets. Additional example IP networks include Voice over IP (VoIP) networks, Service over IP (SoIP) networks, and the like.

In one example, the system 100 may comprise a network 102, e.g., a telecommunication service provider network, a core network, an enterprise network comprising infrastructure for computing and communications services of a business, an educational institution, a governmental service, or other enterprises. The network 102 may be in communication with one or more access networks 120 and 122, and the Internet (not shown). In one example, network 102 may combine core network components of a cellular network with components of a triple play service network; where triple-play services include telephone services, Internet services and television services to subscribers. For example, network 102 may functionally comprise a fixed mobile convergence (FMC) network, e.g., an IP Multimedia Subsystem (IMS) network. In addition, network 102 may functionally comprise a telephony network, e.g., an Internet Protocol/Multi-Protocol Label Switching (IP/MPLS) backbone network utilizing Session Initiation Protocol (SIP) for circuit-switched and Voice over Internet Protocol (VoIP) telephony services. Network 102 may further comprise a broadcast television network, e.g., a traditional cable provider network or an Internet Protocol Television (IPTV) network, as well as an Internet Service Provider (ISP) network. In one example, network 102 may include a plurality of television (TV) servers (e.g., a broadcast server, a cable head-end), a plurality of content servers, an advertising server (AS), an interactive TV/video on demand (VoD) server, and so forth.

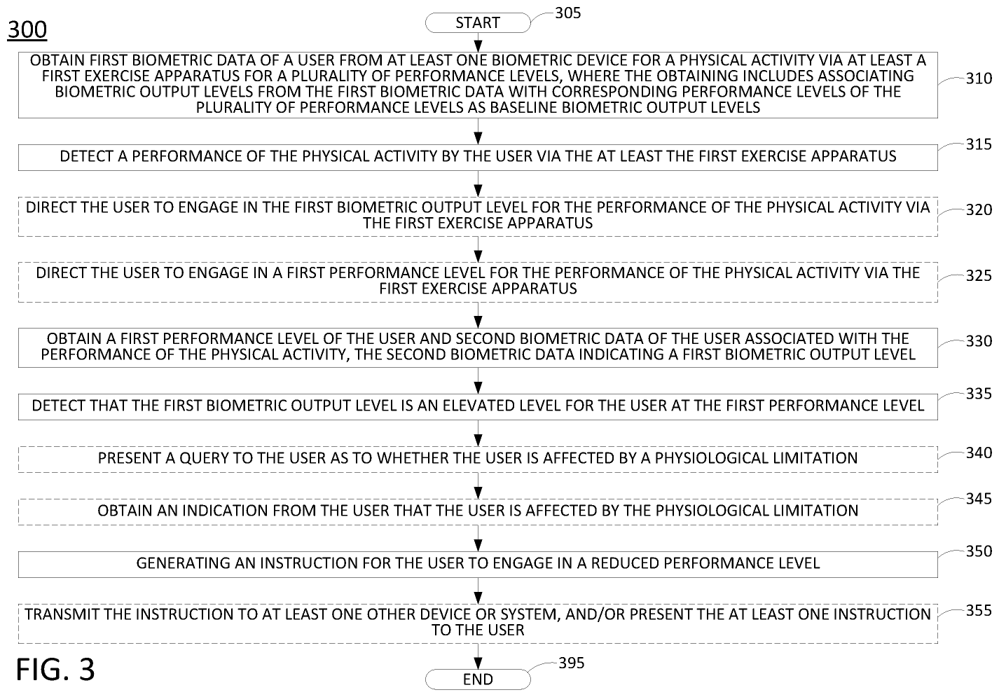

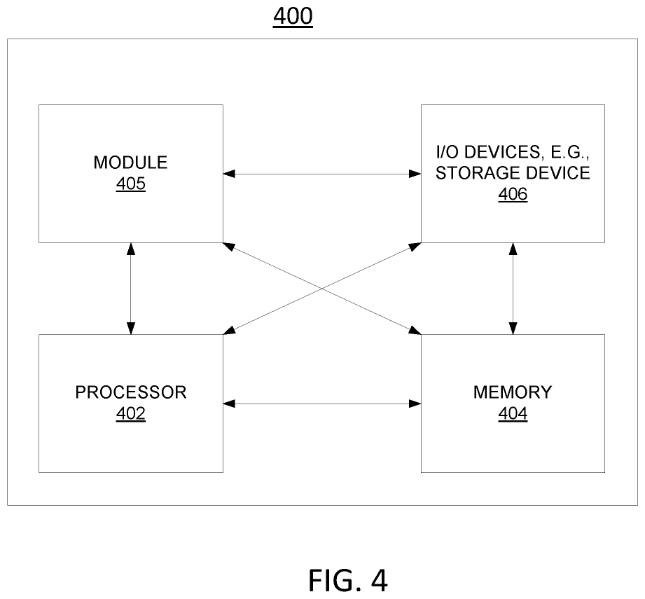

In accordance with the present disclosure, application server (AS) 104 may comprise a computing system or server, such as computing system 400 depicted in FIG. 4, and may be configured to provide one or more operations or functions for generating an instruction for a user to engage in a reduced performance level in response to detecting that a biometric output level of the user for a first physical activity via a first exercise apparatus is elevated as compared to a baseline biometric output level at a first performance level, such as illustrated and described in connection with the example method 300 of FIG. 3. It should be noted that as used herein, the terms “configure,” and “reconfigure” may refer to programming or loading a processing system with computer-readable/computer-executable instructions, code, and/or programs, e.g., in a distributed or non-distributed memory, which when executed by a processor, or processors, of the processing system within a same device or within distributed devices, may cause the processing system to perform various functions. Such terms may also encompass providing variables, data values, tables, objects, or other data structures or the like which may cause a processing system executing computer-readable instructions, code, and/or programs to function differently depending upon the values of the variables or other data structures that are provided. As referred to herein a “processing system” may comprise a computing device including one or more processors, or cores (e.g., as illustrated in FIG. 4 and discussed below) or multiple computing devices collectively configured to perform various steps, functions, and/or operations in accordance with the present disclosure.

Thus, although only a single application server (AS) 104 is illustrated, it should be noted that any number of servers may be deployed, and which may operate in a distributed and/or coordinated manner as a processing system to perform operations for generating an instruction for a user to engage in a reduced performance level in response to detecting that a biometric output level of the user for a first physical activity via a first exercise apparatus is elevated compared to a baseline biometric output level at a first performance level, in accordance with the present disclosure. In one example, AS 104 may comprise a physical activity monitoring server as described herein. In one example, AS 104 may comprise a physical storage device (e.g., a database server), to store various types of information in support of the present example(s). For instance, AS 104 may store user device data (e.g., identifying which devices may be used to communicate with a user, such as device 141 (e.g., smart glasses), device 144 (e.g., a smartwatch), etc., biometric devices/sensors associated with a user (e.g., device 144, a chest-worn heart rate monitor (not shown), or the like), user biometric data and performance data (e.g., baseline data that may match biometric output level(s) with performance level(s), and in one example, biometric output level(s) matched with performance level(s) for particular physical activities, user maximum performance levels (e.g., top running speed (sprinting), maximum weight for bicep curl, maximum torso rotation for trunk exercises, etc.)), information regarding exercise apparatuses (e.g., apparatus type, network address, controllable/adjustable parameters (such as maximum and minimum weight, maximum and minimum resistance, top speed, top incline, etc.), and/or information regarding any integrated biometric sensors), and so forth that may be processed by AS 104 in connection with examples of the present disclosure. For ease of illustration, various additional elements of network 102 are omitted from FIG. 1.

In one example, access network(s) 120 may be in communication with one or more devices or systems including network-based and/or peer-to-peer communication capabilities, e.g., device 141, treadmill 142, device 144, stationary cycle 145, resistance machine 146, and/or controller 149. In one example, activity venue 140 may comprise a home of user 1, a gym that is frequented by user 1, or the like. In one example, various devices or systems in activity venue 140 may communicate directly with one or more components of access network(s) 120 and 122. In another example, controller 149 may be in communication with one or more components of access network(s) 120 and with device 141, treadmill 142, device 144, stationary cycle 145, and/or resistance machine 146, and may send instructions to, communicate with, or otherwise control these various devices or systems. Similarly, the access network(s) 122 may be in communication with one or more devices, such as device 144, smart shoes 147, etc. It should be noted that the same user 1 is illustrated in connection with the use of various exercise apparatuses. Thus, it should be understood that the user 1 may be wearing the same device 144 in each example. In one example, various devices or systems in activity venue 140, as well as smart shoes 147, may communicate with each other via wireless and/or non-wireless peer-to-peer communications, such as Institute for Electrical and Electronics Engineers (IEEE) 802.15 based communications, e.g., Bluetooth, ZigBee, and so forth. For instance, each such device or system may include one or more radio frequency (RF) transceivers for cellular communications and/or for non-cellular wireless communications. In accordance with the present disclosure, controller 149 may also comprise a computing system or server, such as computing system 400 depicted in FIG. 4, and may be configured to provide one or more operations or functions for generating an instruction for a user to engage in a reduced performance level in response to detecting that a biometric output level of the user for a first physical activity via a first exercise apparatus is elevated compared to a baseline biometric output level at a first performance level, such as illustrated and described in connection with the example method 300 of FIG. 3.

As illustrated in FIG. 1, device 144 may comprise a biometric sensor device, such as a smartwatch, e.g., a wireless-enabled wristwatch equipped with a sensor to detect electrocardiogram (ECG/EKG) data, pulse/heartrate data, breathing rate data, perspiration amount, pupil dilation, blood oxygen saturation level data, cholesterol data, sleep/wake data, blood pressure data, movement data (e.g., number of steps, number of pedals, etc.), or the like. Although only a single device 144 is illustrated for collecting biometric data, it should be understood that in another example, different types of biometric data may be collected from multiple wearable biometric devices of user 1 (e.g., such as a smart headband, a smart armband, a smart legband, body-worn smart clothing with embedded sensors, etc.). In one example, smart shoes 147 may include sensors embedded in the soles to measure a number of strides, stride length, duration of ground contact, contact pressure, and so on.

In one example, each of the devices 141 and 144 may comprise any single device or combination of devices that may comprise a user endpoint device. In one example, each of the devices 141 and 144 may include one or more radio frequency (RF) transceivers for cellular communications and/or for non-cellular wireless communications. In addition, in one example, devices 141 and 144 may each comprise programs, logic or instructions to perform operations in connection with examples of the present disclosure for generating an instruction for a user to engage in a reduced performance level in response to detecting that a biometric output level of the user for a first physical activity via a first exercise apparatus is elevated as compared to a baseline biometric output level at a first performance level, such as illustrated and described in connection with the example method 300 of FIG. 3. For example, devices 141 and 144 may each comprise a computing system or device, such as computing system 400 depicted in FIG. 4.

As illustrated in FIG. 1, the device 141 may comprise a wearable computing device (e.g., smart glasses or an augmented reality (AR) headset) and may provide a user interface for user 1. For instance, device 141 may comprise smart glasses or goggles with AR enhancement capabilities. For example, device 141 may have a screen and a reflector to project outlining, highlighting, or other visual markers to the eye(s) of user 1 to be perceived in conjunction with the surroundings. In one example, device 141 may also comprise an outward facing camera to capture video of the physical environment from a field of view in a direction that user 1 is looking. Similarly, device 141 may further include a microphone for voice command/audio inputs. Device 141 may also measure, record, and/or transmit data related to movement and position, such as locations, orientations, accelerations, and so forth. For instance, device 141 may include a Global Positioning System (GPS) unit, a gyroscope, a compass, one or more accelerometers, and so forth.

Access networks 120 and 122 may transmit and receive communications between such devices/systems, and application server (AS) 104, other components of network 102, devices reachable via the Internet in general, and so forth. In one example, the access networks 120 and 122 may comprise Digital Subscriber Line (DSL) networks, public switched telephone network (PSTN) access networks, broadband cable access networks, Local Area Networks (LANs), wireless access networks (e.g., an IEEE 802.11/Wi-Fi network and the like), cellular access networks, 3^{rd} party networks, and the like. For example, the operator of network 102 may provide a cable television service, an IPTV service, or any other types of telecommunication service to subscribers via access networks 120 and 122. In one example, the access networks 120 and 122 may comprise different types of access networks, may comprise the same type of access network, or some access networks may be the same type of access network and others may be different types of access networks. In one example, the network 102 may be operated by a telecommunication network service provider. The network 102 and the access networks 120 and 122 may be operated by different service providers, the same service provider or a combination thereof, or may be operated by entities having core businesses that are not related to telecommunications services, e.g., corporate, governmental or educational institution LANs, and the like. For instance, in one example, one of the access network(s) 122 may be operated by or on behalf of activity venue 140. In one example, each of the access networks 120 and 122 may include at least one access point, such as a cellular base station (e.g., base station 117), a non-cellular wireless access point, a digital subscriber line access multiplexer (DSLAM), a cross-connect box, a serving area interface (SAI), a video-ready access device (VRAD), or the like, for communication with different entities of the system 100.

In an illustrative example, user 1 may enroll in a physical activity monitoring service, e.g., via AS 104. For instance, user 1 may enroll by installing and configuring an application (app) on device 141 and/or device 144 that is in communication with AS 104. In one example, user 1 may input the user's static or relatively static biometric information, such as age, gender, height, weight, current or past injuries, any physiological conditions (e.g., diabetes, high blood pressure, heart conditions, etc.), current physical condition (e.g., as self-assessed by user 1, such as “very fit,” “fit,” “somewhat fit,” “relatively inactive,” “sedentary,” etc.), and so forth. In one example, user 1 may also input one or more health/fitness goals, such as run 5 kilometers in less than 45 minutes, bench press 185 pounds 8 times, achieve a body mass index (BMI) of under 25, and so forth. In one example, user 1 may alternatively or additionally input one or more preferred types of physical activity and/or exercise apparatuses that are available to user 1. For instance, user 1 may wish to engage in a mix of weightlifting/resistance exercises and running for cardiovascular health. However, in one example, user 1 may dislike cycling and may not prefer to use a stationary cycle 145.

In one example, AS 104 may provide a guided fitness program to user 1. For instance, in one example AS 104 may direct user 1 to engage physical activities via different exercise apparatuses to obtain baseline measurements of biometric output levels versus performance levels. For instance, user 1 may be directed (e.g., via communication to device 141 and/or device 144) to run on treadmill 142 at several different speeds. In one example, AS 104 may direct treadmill 142 to engage in different speeds by changing the speed of a roller. For instance, AS 104 may send instructions to treadmill 142 directly and/or via controller 149. At the same time, device 144 may record measurements of the biometric data of user 1 while running at the various speeds (e.g., heart rate, breathing rate, skin temperature, blood oxygen saturation level, etc.) and report the data to AS 104 (e.g., directly, or via device 141 and/or controller 149, etc.). Similarly, user 1 may be directed to engage in exercises via resistance machine 146, where AS 104 may further provide instructions to resistance machine 146 to adjust the resistances levels. For instance, user 1 may be instructed to perform a maximum number of repetitions at 100 pounds of resistance and after 3 minutes of rest, to perform a maximum number of repetitions at 120 pounds of resistance, and so forth. Again, device 144 may record measurements of the biometric data of user 1 while user 1 is engaging in the resistance exercise at various resistance levels (e.g., heart rate, breathing rate, skin temperature, blood oxygen saturation level, perspiration level, pupil dilation, etc.) and report the data to AS 104. In addition, resistance machine 146 may also report the performance level(s) (e.g., number of repetitions achieved) to AS 104.

Additional baseline measurements may be obtained by instructing user 1 to engage via stationary cycle 145, changing the resistance levels, and recording corresponding biometric data (e.g., biometric output levels). In another example, user 1 may be instructed to run at different paces while wearing smart shoes 147, with device 144 recording and reporting corresponding biometric data (biometric output levels). For instance, user 1 may be instructed to “speed up,” “hold pace,” “slow down,” etc., e.g., by way of instructions from AS 104 presented via device 141 and/or device 144, such as an audio output, a message presented via a display of device 141 and/or device 144, etc. However, it should be noted that in another example, user 1 may alternatively or additionally engage in a self-selected exercise regimen, wherein device 144 may report biometric data, and wherein different exercise equipment may report corresponding performance levels (or settings that may be associated with performance levels) to AS 104. For instance, AS 104 may match time-stamped biometric data with time-stamped performance levels to be stored as baseline metrics. In other words, the baseline measurements may merely be assessed when user 1 is going through his or her regular exercise routines. In one example, data may be collected over a period of time (e.g., a week, two-weeks, etc.) and/or over multiple sessions of physical activities, where baseline metrics may be calculated from averages of biometric output levels and/or performance levels. Alternatively, or in addition, baseline metrics may be taken as the top performance level(s) achieved (e.g., overall, for a given biometric output level, etc.), and so forth.

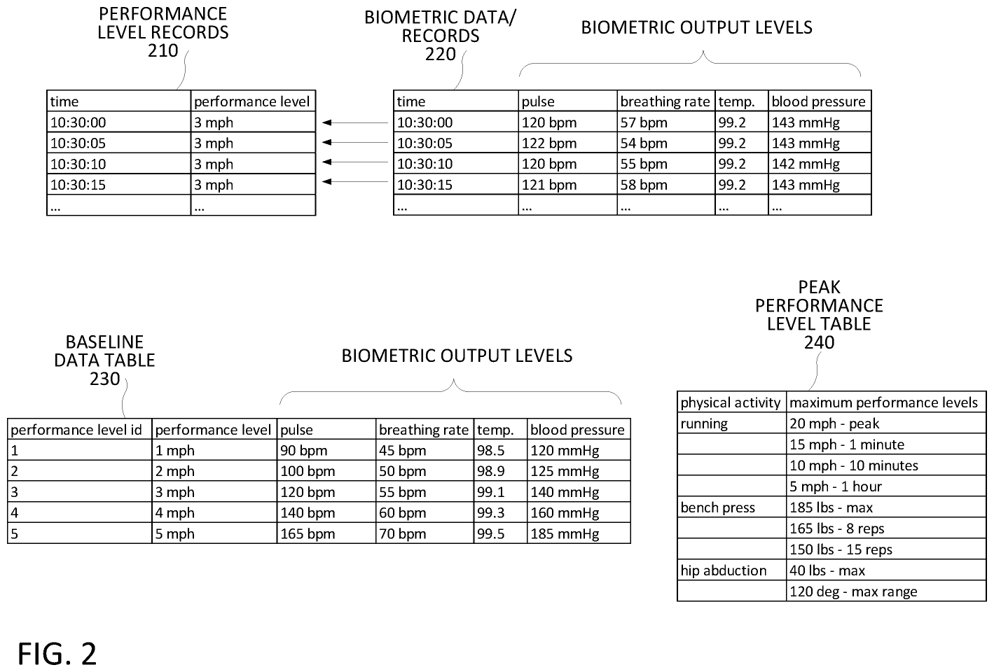

In this regard, FIG. 2 illustrates an example table (e.g., baseline data table 230) containing baseline metrics. For instance, FIG. 2 further depicts an example table 210 of performance level records and an example table 220 of biometric data records. Notably, each of the performance level records of table 210 may include a time (or “timestamp”) and a corresponding performance level as measured via an exercise apparatus. In this case, the example records may relate to a treadmill, such as treadmill 142 of FIG. 1. In addition, in the present example, it appears that a user (e.g., user 1 of FIG. 1) has been maintaining a steady performance level (pace) of 3 mph. It should be noted that for illustrative purposes, performance levels may be segregated into 1 mph increments. For instance, actual paces of 2.5 mph to 3.4 mph may be considered as a “3 mph” performance level. However, in other, further, and different example, different gradations of performance levels may be utilized, such as each 0.5 mph increment being a performance level, etc. The example table 220 of biometric data records may also include, for each row/record, a time/timestamp and one or more corresponding biometric output levels for one or more types of biometric data, e.g., in the present example: pulse, breathing rate, temperature, blood pressure.

In one example, in order to obtain baseline metrics, the present disclosure may match measured biometric output levels to the corresponding performance levels. For instance, this may be achieved by matching records from table 210 with records from table 220 having the same timestamps. As noted above, in one example, a baseline of biometric output levels for a given performance level may be obtained by averaging measured biometric output levels for the given performance level over multiple records. For instance, in this case, each of the biometric data records in table 220 may be associated with a performance level of 3 mph. As such, the biometric output levels may be averaged for each type of biometric data, respectively. In the present example, the result may be a baseline record in table 230 for the performance level of “3 mph.” For instance, the average pulse/heart rate for the user at 3 mph is 120 beats per minute (bpm). Similar measures are indicated for breathing rate, temperature, and blood pressure, respectively. In addition, the record also includes a performance level identifier (id), which may be used for purposes of addressing the record in the table 230. Table 230 includes additional records for four other performance levels, each of which may be generated in the same or similar manner as described above.

As also mentioned above, in one example, the present disclosure may further baseline peak performance levels that the user may achieve. Thus, for example, table 240 illustrates peak performance levels of a user (such as user 1 of FIG. 1) with respect to three different physical activities. It should be noted that each activity may have one or more peak performance levels. In addition, the total number of peak performance levels may vary from activity to activity, e.g., depending upon the nature of the activity, the number of settings of the exercise apparatus that may be available, and so forth. For instance, with respect to running, user 1 may be able to achieve a top speed of 20 mph. However, the user may not be able to sustain this pace. On the other hand, user 1 may also have demonstrated an ability to maintain a speed of 15 mph over the course of at least 1 minute. Table 240 also illustrates that for some physical activities, such as hip abduction, a peak performance level may comprise a maximum range of motion. For instance, user 1 may have demonstrated an ability to open hips up to 120 degrees. However, other users may have less flexibility and may only reach a maximum of 110 degrees, for instance, as measured by an exercise apparatus during use (e.g., a hip abduction resistance machine, or the like). In one example, the baseline data table 230 and the peak performance level table 240 of FIG. 2 may thus be generated, and stored by AS 104 of FIG. 1 for use in detecting deviations of biometric output level(s) for given performance level(s).

In one example, after obtaining baseline measurements, AS 104 may then provide directed exercise/physical activities to user 1 or may monitor user 1 when user 1 engages in various physical activities. For instance, in a directed fitness program, AS 104 may obtain an input indicating that user 1 is ready to commence a physical activity session. In one example, user 1 may indicate an amount of time available, and AS 104 may tailor instructions to such quantity of time. In another example, AS 104 may provide an advance plan to user 1 indicating an amount of time that is anticipated to be needed to complete a plan for a next/current session. By starting the plan, it may be assumed that user 1 has sufficient time to complete the plan. In any case, AS 104 may direct the user to a particular activity. For instance, user 1 may be directed to treadmill 142 in order to walk, run, jog, etc. In addition, user 1 may be instructed to achieve one of: a first performance level and/or a first biometric output level for the performance of the physical activity via the first exercise apparatus. To illustrate, in one example, user 1 may be instructed to run at a pace of 5 mph. In another example, user 1 may be instructed to reach and maintain a heart rate of around 150 bpm.

In another example, AS 104 may provide semi-supervised guidance to user 1. For instance, user 1 may indicate to AS 104 an intention to use a particular exercise apparatus. AS 104 may then direct user 1 as to a recommended performance level (e.g., a resistance, weight, speed, etc.) and/or a recommended biometric output level (e.g., a target heart rate or the like). In still another example, AS 104 may not provide a directed fitness program to user 1, but may still monitor for possible physiological impairments. For instance, user 1 may commence the use of a particular exercise apparatus (e.g., treadmill 142) and may engage at a performance level of the user's choosing.

In each such case, AS 104 may collect biometric data of user 1 (e.g., from device 144) during the performance of the corresponding physical activity (e.g., running on treadmill 142). The biometric data may include one or more “biometric output levels,” e.g., a heart rate, a breathing rate, a skin temperature, a blood oxygen saturation level, a blood pressure level, and so forth. In addition, AS 104 may collect performance level data (e.g., from treadmill 142, if not implicit in prior instructions from AS 104 to the treadmill 142). AS 104 may monitor the physical activity (and for subsequent physical activities) on an ongoing basis to detect whether a biometric output level is elevated for the user at a corresponding performance level. For instance, blood pressure may typically increase with vigorous exercise, which may be captured in the baseline data for the user (e.g., blood pressure of an average of X when running at speed A, blood pressure of an average of Y when running at speed B, etc.). In an illustrative example, user 1 may be directed to run at a speed of 5 mph on treadmill 142. The blood pressure may be currently measured at 210 mmHg via device 144, whereas the baseline for user 1 may be 180 mmHg at 5 mph. In one example, a spike of 15 percent or more may be considered “elevated” as compared to the baseline (e.g., 207 mmHg or more). Accordingly, in this case, AS 104 may determine that user 1 has at least one elevated biometric output level for the given performance level of 5 mph.

Notably, AS 104 may not determine any particular underlying cause. However, given that user 1 is typically able to achieve a 5 mph running pace with blood pressure of 180 mmHg, but is presently exerting at 210 mmHg, AS 104 may conclude that user 1 is potentially affected by a new physiological condition/impairment that is preventing the achievement of a typical physical performance. For instance, a possible cause may be that the user has recently eaten, or drank one or more caffeinated beverages and/or alcoholic beverages. Another possible cause may be an injury to the user that causes the user to have a shortened stride, limp, or the like, such that the user must work harder to achieve a 5 mph pace than if the user were fully healthy. It should be noted that some users may attempt to work through injuries to their own detriment. Nevertheless, AS 104 may indirectly detect these scenarios via comparison of biometric output levels for given performance levels as compared to stored baseline data.

In one example, upon detection of a biometric output level being elevated for user 1 at a corresponding performance level, AS 104 may implement a remedial action. In particular, AS 104 may direct user 1 to engage in a reduced performance level via treadmill 142 (e.g., do not exceed 3 mph). Alternatively, or in addition, when user 1 attempts to engage a different exercise apparatus, such as stationary cycle 145, user 1 may be directed to engage in a similarly reduced performance level. For instance, in addition to mapping biometric output levels to performance levels to baseline particular activities, AS 104 may further establish associations between similar performance levels across different activities. For example, if user 1 exhibits an average heart rate of 160 bpm when running at 5 mph and similarly exhibits an average heart rate of 160 bpm when cycling at 16 mph (e.g., at a resistance level “2” out of “5”), AS 104 may associate these two performance levels as being the same and/or correspondent with one another. AS 104 may similarly associate higher performance levels (e.g., running at 6 mph, cycling at 20 mph) and lower/lesser performance levels (e.g., running at 3 mph, cycling at 10 mph). Accordingly, if it is detected via one modality (e.g., treadmill 142) that user 1 may have a new physiological impairment, user 1 may be directed to engage in a lesser performance level via treadmill 142 (e.g., not to exceed 3 mph), and may additionally be directed to engage in a lesser performance level via stationary cycle 145 (e.g., not to exceed 10 mph). In another example, a machine that is helping a user to perform physical therapy or isometric stretching for flexibility training, such as a rowing machine or wall-climb simulator, may establish different amounts distance displacement for a stretch of legs or arms for user 1. In yet another example, a device that is similarly used for therapeutic restoration of muscles or flexibility, such as assisted curls or leg lefts, may establish or detect a maximal angle that an arm or leg should be contracted or extended.

In one example, a potential new physiological impairment may be determined when two or more biometric output levels are detected to be elevated for a given performance level. For instance, if user 1 is running at 5 mph on treadmill 142, and the blood pressure is detected to be 210 mmHg and the heart rate is detected to be 175 bpm, these two different factors may cause AS 104 to determine that a physiological impairment is likely (and to take corresponding remedial action(s)). For instance, when a heart rate measure exceeds a baseline for a particular performance level by 5 percent (or similar threshold), AS 104 may determine that the heart rate is “elevated.”

In one example, AS 104 may not immediately direct user 1 to engage in a reduced performance level. Rather, AS 104 may present user 1 with its conclusion that a physiological impairment is likely. For instance, AS 104 may present the performance level, the corresponding biometric output level(s) as measured, and the baseline of biometric output level(s) for the particular performance level. AS 104 may also present a recommended reduced performance level (and in one example, the predicted corresponding biometric output level(s)) for user 1 to agree to (or to dismiss). For instance, AS 104 may send instructions to device 141 and/or device 144 to present an audio and/or visual output to the user with the foregoing information. User 1 may then speak a voice command/response and/or provide a touch input, etc. to indicate “I agree” or “dismiss.” In one example, a voice dialog or visual user interface may enable user 1 to provide an input specifying a particular physiological impairment. For instance, user 1 may input “muscle strain—legs,” “muscle strain—abdominals,” “bruising,” “recent meal,” etc. to indicate a potential root cause. In such case, AS 104 may adjust an exercise regimen accordingly. For instance, for a muscle strain, AS 104 may direct reduced performance levels for one or more physical activities over a 3 week period. However, for a “recent meal” AS 104 may direct reduced performance levels for only the next 2 hours, or the like.

It should be noted that AS 104 may provide different features when it is detected that biometric output levels are not elevated for user 1 at particular performance levels. For instance, user 1 may be directed to run at 5 mph on treadmill 142. However, AS 104 may detect via communication from treadmill 142 that user 1 has only reached 4 mph. In one example, the user may be detected to have a biometric output level that corresponds to a typical pace of the user at 5 mph, thus indicating an elevated biometric output level for the corresponding current performance level of 4 mph. In other words, user 1 may be unable to achieve the directed 5 mph pace and may stop at 4 mph, e.g., due to an underlying physiological impairment. In such case, AS 104 may direct the user to engage in a further reduced performance level (e.g., not to exceed 3 mph) or may direct the user to maintain 4 mph (e.g., to no longer attempt to achieve the target speed of 5 mph).

However, in another illustrative example, AS 104 may determine from biometric data from device 144 and speed data from treadmill 142 that although user 1 is running at 4 mph and has not achieved the target speed of 5 mph, the user's heart rate, blood pressure, etc. are “normal” for the 4 mph performance level (e.g., are matched to or within a tolerance range (e.g., 5 percent, 10 percent, etc.) of the baseline). In this case, AS 104 may conclude that there is no apparent physiological impairment. In one example, AS 104 may further present user 1 with one or more motivational messages when the user 1 has not achieved a target/directed performance level and there is no disconnect between the biometric output level(s) and the corresponding performance level(s) being achieved. For instance, AS 104 may send one or more instructions to device 141 and/or device 144 to present one or more audible messages to user 1, such as “you are off pace,” “speed it up,” “you can do it,” etc.

The foregoing describes several illustrative scenarios in which examples of the present disclosure for generating an instruction for a user to engage in a reduced performance level in response to detecting that a biometric output level of the user for a first physical activity via a first exercise apparatus is elevated as compared to a baseline biometric output level at a first performance level may operate in connection with the example system 100. Thus, it should be understood that in various examples, other, further, and different arrangements may be deployed in accordance with the present disclosure. For instance, operations described above with respect to AS 104 may alternatively or additionally be performed by controller 149. For example, controller 149 may collect and store baseline performance and biometric data, may control/instruct treadmill 142, stationary cycle 145, and/or resistance machine 146 to implement different controllable settings to provide specified performance levels, or to place limits on such controllable settings to correspondingly limit performance levels to one or more maximum performance levels (e.g., maximum resistance of 200 pounds, speed not to exceed 12 mph, etc.), may communicate with user 1 via devices 141 and/or 144 to establish one or more exercise regimens, to direct user 1 to engage in one or more specified performance levels and/or biometric output levels, to report detected potential physiological impairments, to obtain user assent to one or more reduced performance levels, and so forth.

Similarly, in still another example, the same or similar operations may be provided by device 141 and/or device 144. For instance, device 144 may be configured to communicate in a peer-to-peer fashion and/or via one or more networks with exercise apparatuses, such as treadmill 142, stationary cycle 145, resistance machine 146, smart shoes 147, etc. In addition, device 144 may obtain and store baseline data such as described above, may monitor ongoing physical activities via the same or different exercise apparatuses to detect that a biometric output level at a given performance level is elevated as compared to a baseline biometric output level at such a performance level, and so forth.

In addition, although the foregoing example(s) is/are described and illustrated in connection with an individual user (user 1) and with a single activity venue 140, it should be noted that various other scenarios may be supported in accordance with the present disclosure wherein multiple users are served by AS 104 and/or controller 149, and/or wherein user 1 may be served by AS 104 and/or controller 149 across multiple venues (e.g., different gyms, a home gym and a public gym, etc.). Thus, these and other modifications are all contemplated within the scope of the present disclosure.

It should also be noted that the system 100 has been simplified. In other words, the system 100 may be implemented in a different form than that illustrated in FIG. 1. For example, the system 100 may be expanded to include additional networks, and additional network elements (not shown) such as wireless transceivers and/or base stations, border elements, routers, switches, policy servers, security devices, gateways, a network operations center (NOC), a content distribution network (CDN) and the like, without altering the scope of the present disclosure. In addition, system 100 may be altered to omit various elements, substitute elements for devices that perform the same or similar functions and/or combine elements that are illustrated as separate devices.

As just one example, as noted above, one or more operations described above with respect to AS 104 may alternatively or additionally be performed by controller 149, and vice versa. In addition, although a single AS 104 is illustrated in the example of FIG. 1, in other, further, and different examples, the same or similar functions may be distributed among multiple other devices and/or systems within the network 102, access network(s) 120 or 122, and/or the system 100 in general that may collectively provide various services in connection with examples of the present disclosure. Additionally, devices that are illustrated and/or described as using one form of communication (such as a cellular or non-cellular wireless communications, wired communications, etc.) may alternatively or additionally utilize one or more other forms of communication. In an alternative example, one of the devices 141 or 144 may comprise a cellular smart phone, a laptop, a tablet computer, or the like. In addition various other exercise apparatuses may be included within the scope of the present disclosure, such as network-connected rowing machines, a pool with jets that may be used to control a flow of water/current and to thus control a swimming speed, a network-connected and/or wireless communication capable bicycle, and so on. In still another example, the device 141 may comprise a virtual reality (VR) headset and may provide a user interface for user 1.

In one example, the user may utilize one or more additional apparatuses in connection with a virtual reality or “metaverse” experience, such as a 360-degree/omnidirectional treadmill, force feedback gloves, and so forth. For instance, a 360-degree treadmill may be controlled similarly to treadmill 142 described above, such as reducing a resistance of a roller pad, reducing speed, reducing incline, etc. In addition, to further illustrate, force feedback gloves may include a plurality of actuators which may be controllable to provide positive force and/or movement of various portions of the hands of the a user (e.g., electromechanical actuators or motors, electro-hydraulic actuators, electro-pneumatic actuators, etc.). Thus, for example, more or less force/resistance and/or movement may be provided via such force feedback gloves depending upon whether a user is determined to be affected by a physiological impairment or not. For instance, force feedback gloves may cause a “virtual object” to be made to as to feel heavy and hence difficult to “throw”. However, if a user is determined to be affected by a physiological impairment, the virtual object may be made to feel lighter/less heavy via control of the actuators and/or motors of the force feedback gloves. In this regard, such devices or the like that are utilized in a VR experience (and/or in an AR experience) may be considered as additional examples of exercise apparatuses. Thus, these and other modifications are all contemplated within the scope of the present disclosure.

FIG. 3 illustrates a flowchart of an example method 300 for generating an instruction for a user to engage in a reduced performance level in response to detecting that a biometric output level of the user for a first physical activity via a first exercise apparatus is elevated as compared to a baseline biometric output level at a first performance level. In one example, steps, functions and/or operations of the method 300 may be performed by a device or apparatus as illustrated in FIG. 1, e.g., by AS 104, controller 149, or any one or more components thereof, or by AS 104, controller 149, and/or any one or more components thereof in conjunction with one or more other components of the system 100, such as a different one or AS 104 or controller 149, device 141, device 144, exercise apparatuses, and so forth. In one example, steps, functions and/or operations of the method 300 may be performed by a user device or apparatus, such as device 141 or device 144, or device 141 or device 144 in conjunction with one or more other components of the system 100, such as a different one of device 141 or device 144, one or more exercise apparatuses, and so forth. In one example, the steps, functions, or operations of method 300 may be performed by a computing device or processing system, such as computing system 400 and/or hardware processor element 402 as described in connection with FIG. 4 below. For instance, the computing system 400 may represent any one or more components of the system 100 that is/are configured to perform the steps, functions and/or operations of the method 300. Similarly, in one example, the steps, functions, or operations of the method 300 may be performed by a processing system comprising one or more computing devices collectively configured to perform various steps, functions, and/or operations of the method 300. For instance, multiple instances of the computing system 400 may collectively function as a processing system. For illustrative purposes, the method 300 is described in greater detail below in connection with an example performed by a processing system. The method 300 begins in step 305 and proceeds to step 310.

At step 310, the processing system obtains first biometric data of a user from at least one biometric device for a physical activity via at least a first exercise apparatus of a first exercise apparatus type for a plurality of performance levels, where the obtaining includes associating biometric output levels from the first biometric data with corresponding performance levels of the plurality of performance levels as baseline biometric output levels. The biometric data may include one or more “biometric output levels” for one or more different types of biometric data, e.g., a heart rate, a breathing rate, a skin temperature, a blood oxygen saturation level, a blood pressure level, and so forth.

In accordance with the present disclosure, the plurality of performance levels may include: levels of speed (e.g., 1 mph, 2 mph, . . . , 5 mph, . . . , 10 mph, etc.), levels of distance (e.g., 50 meters, 100 meters, 1000 meters, 1 mile, 5 kilometers, 10 miles, etc.), levels of time of performance (e.g., 30 seconds, 1 minute, 2 minutes, 5 minutes, 30 minutes, 1 hour, etc.), levels of repetitions per weight and/or per resistance (e.g., 8 repetitions (reps) at 20 pounds, 15 reps at 2 pounds, 4 repetitions at 100 pounds, 15 repetitions at 30 pounds, etc.), and so forth. In addition, the plurality of performance levels may include maximum levels per physical activity, which may include maximum speed, maximum weight (e.g., for 1 rep), maximum resistance (e.g., for 1 rep), etc.

Each physical activity level may be associated with one or more corresponding biometric output levels. To illustrate, 8 repetitions at 100 pounds may result in a first set of biometric output levels, while 15 repetitions at 100 pounds may result in a second set of biometric output levels (e.g., measured as the peak measurement during the exercise or within say 30 seconds of completion). It should also be noted that the user may not be able to complete 15 repetitions. Rather, a maximum for a particular weight or resistance may be 12, which may be recorded in the performance level data (and which may be further associated with one or more biometric output levels (e.g., for one or more different types of biometric data that is/are available). In one example, the one or more biometric output levels may be average levels of biometric measurements for one or more different types of biometric data for the user performing a same physical activity at a same performance level (e.g., an average pulse/heart rate of the user when running at 5 mph as measured over several running sessions on one or more treadmills). In one example, step 310 may include obtaining performance level data from one or more exercise apparatuses, e.g., time-stamped records indicating the performance level of the user, such as 10:00:00 AM 5.0 mph, 10:00:10 AM 4.9 mph, 10:10:20 AM 5.1 mph, etc.

In one example, the performance levels may be associated with biometric data (e.g., biometric output levels) by matching time-stamped biometric data (e.g., biometric output levels) with time stamped performance level data (e.g., matching those records having the same timestamps). However, as noted above, in one example, the one or more biometric output levels may be average levels of biometric measurements for one or more different types of biometric data for the user performing a same physical activity at a same performance level. Thus, the biometric output levels of the final baseline data may be aggregated over multiple biometric data records.

At step 315, the processing system detects a performance of the physical activity by the user via the at least the first exercise apparatus. For instance, the user may indicate a use of the first exercise apparatus via a near-field communication (NFC) detection and/or pairing of an endpoint device of the user and the first exercise apparatus (or similarly a radio-frequency identification (RFID) tag of the user, or the like), via a user input of a unique code associated with the user and identifying the user to the first exercise apparatus, via the user scanning a barcode, quick response (OR) code or the like of the first exercise apparatus using a camera of an endpoint device of the user (and subsequently communicating the detected code to the processing system), and so forth. Alternatively, or in addition, the processing system may detect performance data being provided by the first exercise apparatus, which may be uniquely associated with the user (e.g., the user's own smart shoes that are not associated with any other users, or the like).

At optional step 320, the processing system may direct the user to engage in the first biometric output level for the performance of the physical activity via the first exercise apparatus. For instance, the processing system may provide an exercise regimen for the user, which may include one or more physical activities that involve maintain a target biometric output level (or levels) for specified time periods and/or durations. The exercise regimen may be generated by the processing system based upon user goals and physiological characteristics (such as age, gender, current/prior fitness level, and so forth). Alternatively, or in addition, the exercise regimen may be selected by the user and preprogrammed into the processing system.

At optional step 325, the processing system may direct the user to engage in a first performance level of the physical activity via the first exercise apparatus. For instance, as noted above the processing system may provide an exercise regimen for the user. In one example, the exercise regimen may include one or more physical activities that involve reaching one or more target performance levels. In one example, optional step 325 may include selecting at least a first setting of the first exercise apparatus, which may include: a first resistance level, a first weight load, a first incline level, a first speed, a first distance displacement, a first maximal angle, etc. It should be noted that the term “first” does not necessarily mean the lowest or highest available setting in a sequence of settings. Rather, the term “first” is used as a label only to distinguish from a “second,” a “third,” etc. of the same type of setting or other settings. Thus, the terms “first,” “second,” “third,” etc. do not necessarily connote a specific order, sequential relationship, relative size or magnitude, or the like, unless otherwise specifically indicated.

At step 330, the processing system obtains performance level data (e.g., first performance level, a second performance level, etc.) of the user and second biometric data of the user associated with the performance of the physical activity, where the second biometric data indicates a first biometric output level. In one example, the processing system may comprise the at least one biometric device and step 330 may include measuring biometric data of the user via the at least one biometric device. In another example, step 330 may include obtaining the second biometric data from the at least one biometric device, where the processing system is distinct from the at least one biometric device. In one example, step 330 may include obtaining performance level data from the first exercise apparatus. For instance, the first exercise apparatus may report its current settings, such as a speed, a weight, a resistance, a range of motion, etc. In one example, the at least one biometric device may be integrated with the first exercise apparatus. In such case, the performance level data and the second biometric data may both be obtained from the first exercise apparatus.

At step 335, the processing system detects that the first biometric output level of the user is an elevated level for the user at a first performance level. For example, step 335 may comprise detecting that the first biometric output level of the user is elevated for the first particular performance level as compared to the baseline (for the performance of the physical activity at the first performance level). For instance, when such a condition is detected, the processing system may determine that the user is underperforming due to a potential physiological condition (e.g., and not a motivational problem). To illustrate, blood pressure of the user may be measured at 210 mmHg via the at least one biometric device. At the same time the performance level may be obtained from the first exercise apparatus, which may be “5 mph.” Notably, the baseline for the user may be 180 mmHg at 5 mph. In one example, a spike of 15 percent or more (or any other preselected percentages) may be considered “elevated” as compared to the baseline (e.g., 207 mmHg or more). Accordingly, in this case, the processing system may determine that the user has at least one elevated biometric output level for the first performance level. In other examples, different thresholds may be used (e.g., 10 percent or more, 18 percent or more, etc.), such as for different types of biometric data, different categories of users (e.g., elite athletes, “weekend warriors,” casual exercisers, etc.), and so forth. In one example, one or more thresholds may be tailored to the user. For instance, the processing system may also track and learn over time a range of variation for the user of biometric output level(s) for a given performance level for a physical activity (and so on for other performance levels for the same physical activity, and similarly for different physical activities).

At optional step 340, the processing system may present, in response to the detecting that the first biometric output level of the user is an elevated level for the user at the first performance level, a query to the user as to whether the user is currently affected by a physiological limitation. For instance, the user may be affected by a physiological limitation that the user may first attempt to hide, mask, ignore, etc. Thus, the user may not proactively input such a physiological limitation into the processing system. However, the user may be willing to concede to such a condition when specifically queried at to the same.

At optional step 345, the processing system may obtain an indication from the user that the user is affected by a physiological limitation. For instance, the first user may acknowledge the physiological limitation in response to a specific query. In one example, the indication from the user may include an identification of the type of physiological limitation, e.g., “muscle strain—leg,” “ear crystals” (which may affect balance), “large meal,” “sprained ankle,” etc.

At step 350, the processing system generates an instruction for the user to engage in a reduced performance level for at least one of: the physical activity via the first exercise apparatus or a performance of at least a second physical activity via a second exercise apparatus, where the reduced performance level is a lesser performance level as compared to the first performance level. In one example, the reduced performance level may be selected for the user in response to the obtaining at optional step 345 of the indication from the user that the user is affected by the physiological limitation. In one example, the processing system may select an extent of the reduction in performance level based upon the type of physiological limitation (e.g., if obtained from the user at optional step 345, in one example).

In one example, step 350 may include selecting at least a second setting of the first exercise apparatus, such as: a second resistance level that is less than a first resistance level, a second weight load that is less than a first weight load, a second incline level that is less than a first incline level, a second speed that is less than a first speed, etc. (e.g., where the first resistance level, first weight load, first incline level, first speed, etc. may be selected at optional step 325 and/or may be currently engaged in by the user at the user's own selection).

In one example, step 350 may comprise mapping the first performance level for the physical activity to a corresponding first performance level for the at least the second physical activity, where the reduced performance level is a lesser performance level as compared to the first performance level for the at least the second physical activity. For instance, the processing system may learn the mappings for multiple performance levels across various physical activities, or may be configured with the mappings. For example, the system may learn that users who are able to engage in the first performance level for the first physical activity at a given biometric output level may also engage in the first performance level for the at least the second physical activity at the same given biometric output level (e.g., a level of exertion). Thus, for a user who is able to perform at a certain performance level at a certain level of exertion (evidenced by the biometric output level) for the first physical activity may be expected to be able to perform at a similar performance level at the same level of exertion for a second physical activity, and so forth for other performance levels of the respective physical activities that may be mapped to each other via sharing of the same biometric output level(s). In one example, the mappings may be user-specific. For instance, the processing system may learn what level of performance can be expected from the user at different levels of exertion for different exercises, where the levels of performance from exercise to exercise are mapped to each other based upon being for a same biometric output level of the user (e.g., for a particular type of biometric data, such as heart rate data, or for a same set of biometric output levels of the user across multiple types of biometric data (e.g., blood pressure and heart rate, which may generally track together)).

In one example, the first physical activity may comprise a first type of resistance exercise or weightlifting exercise and the at least the second physical activity may comprise at least a second type of resistance exercise or weightlifting exercise. In one example, the reduced performance level may be a percentage drop or absolute drop in weight and/or resistance from a planned/scheduled level, e.g., according to an exercise regimen for the user. For instance, the reduced performance level may be a 30% reduction in weight and/or resistance, a 40% reduction in weight and/or resistance, a 50 lb. reduction in and/or resistance, etc.

At optional step 355, the processing system may transmit the instruction to at least one other device or system, and/or present the at least one instruction to the user. For instance, in one example, the processing system may be a processing system of an endpoint device of the user and optional step 355 may comprise presenting the instruction for the user, e.g., as an audio output or a visual output. For instance, the processing system may comprise a user endpoint device such as an augmented reality headset (e.g., smart glasses), a smartwatch, etc. Alternatively, or in addition, optional step 355 may comprise transmitting the instruction to an endpoint device of the user (e.g., where the processing system may comprise a network-based server, or the like). In one example, optional step 355 may include transmitting the instruction to the first exercise apparatus to directly implement the reduced performance level via at least one setting of the first exercise apparatus. In one example, optional step 355 may include transmitting the instruction to the second exercise apparatus to provide the reduced performance level via at least one setting of the second exercise apparatus. In one example, the instruction may indicate the reduced performance level, and the first or second exercise apparatus may automatically select the appropriate corresponding settings. Alternatively, the instruction can specify the setting(s) to provide the reduce performance level, which may be implemented by the first or second exercise apparatus. It should be noted that in one example, the instruction may be transmitted to different entities, such as to an endpoint device of the user to present an audio or visual output, and to the first or second exercise apparatus to implement one or more settings to effect the reduced performance level.

Following step 350 or optional step 355, the method 300 proceeds to step 395. At step 395, the method 300 ends.

It should be noted that the method 300 may be expanded to include additional steps, or may be modified to replace steps with different steps, to combine steps, to omit steps, to perform steps in a different order, and so forth. For instance, in one example, the processing system may repeat one or more steps of the method 300, such as performing steps 310-350 or steps 310-355 on an ongoing basis for the user. In one example, the processing system may repeat steps 315-330, e.g., until it is detected that a biometric output level of the user is at an elevated level for the user at a corresponding performance level. In one example, the processing system may repeat steps 315-330, steps 315-350, and/or steps 315-355 for different physical activities of the user via the first exercise apparatus or one or more other exercise apparatuses.

In another example, the method 300 may alternatively or additionally comprise detecting that the user is performing at a lesser performance level than the first performance level, detecting that the first biometric output level matches the first performance level, and generating an instruction, e.g., directing the user to engage in a reduced performance level for at least one of: the performance of the physical activity via the first exercise apparatus or a performance of at least a second physical activity via a second exercise apparatus, where the reduced performance level is a lesser performance level of the plurality of performance levels as compared to the first performance level. For instance, the user may never reach the first performance level, but the user's heart rate, breathing rate, etc. may match such performance level (e.g., the user is working hard but not getting to where the user normally would, which is indicative of a potential physiological condition, rather than a motivational issue). In still another example, the method 300 may alternatively or additionally comprise detecting that the user is performing at a lesser performance level than the first performance level, detecting that the first biometric output level matches the lesser performance level, and providing a motivational message to the user. For instance, in this case, the user does not appear to be overexerted for the detected performance level. In such case, it may be determined by the processing system that the user did not likely suffer from a potential physiological condition, but is instead underperforming (e.g., is not sufficiently motivated to perform at a higher level) and may benefit from further motivation. In various other examples, the method 300 may further include or may be modified to comprise aspects of any of the above-described examples in connection with FIGS. 1 and 2, or as otherwise described in the present disclosure. Thus, these and other modifications are all contemplated within the scope of the present disclosure.

In addition, although not expressly specified above, one or more steps of the method 300 may include a storing, displaying and/or outputting step as required for a particular application. In other words, any data, records, fields, and/or intermediate results discussed in the method 300 can be stored, displayed and/or outputted to another device as required for a particular application. Furthermore, operations, steps, or blocks in FIG. 3 that recite a determining operation or involve a decision do not necessarily require that both branches of the determining operation be practiced. In other words, one of the branches of the determining operation can be deemed as an optional step. However, the use of the term “optional step” is intended to only reflect different variations of a particular illustrative embodiment and is not intended to indicate that steps not labelled as optional steps to be deemed to be essential steps. Furthermore, operations, steps or blocks of the above described method 300 can be combined, separated, and/or performed in a different order from that described above, without departing from the example embodiments of the present disclosure.

FIG. 4 depicts a high-level block diagram of a computing system 400 (e.g., a computing device or processing system) specifically programmed to perform the functions described herein. For example, any one or more components, devices, and/or systems illustrated in FIG. 1 or described in connection with FIG. 2 or 3, may be implemented as the computing system 400. As depicted in FIG. 4, the computing system 400 comprises a hardware processor element 402 (e.g., comprising one or more hardware processors, which may include one or more microprocessor(s), one or more central processing units (CPUs), and/or the like, where the hardware processor element 402 may also represent one example of a “processing system” as referred to herein), a memory 404, (e.g., random access memory (RAM), read only memory (ROM), a disk drive, an optical drive, a magnetic drive, and/or a Universal Serial Bus (USB) drive), a module 405 for generating an instruction for a user to engage in a reduced performance level in response to detecting that a biometric output level of the user for a first physical activity via a first exercise apparatus is elevated as compared to a baseline biometric output level at a first performance level, and various input/output devices 406, e.g., a camera, a video camera, storage devices, including but not limited to, a tape drive, a floppy drive, a hard disk drive or a compact disk drive, a receiver, a transmitter, a speaker, a display, a speech synthesizer, an output port, and a user input device (such as a keyboard, a keypad, a mouse, and the like).

Although only one hardware processor element 402 is shown, the computing system 400 may employ a plurality of hardware processor elements. Furthermore, although only one computing device is shown in FIG. 4, if the method(s) as discussed above is implemented in a distributed or parallel manner for a particular illustrative example, e.g., the steps of the above method(s) or the entire method(s) are implemented across multiple or parallel computing devices, then the computing system 400 of FIG. 4 may represent each of those multiple or parallel computing devices. Furthermore, one or more hardware processor elements (e.g., hardware processor element 402) can be utilized in supporting a virtualized or shared computing environment. The virtualized computing environment may support one or more virtual machines which may be configured to operate as computers, servers, or other computing devices. In such virtualized virtual machines, hardware components such as hardware processors and computer-readable storage devices may be virtualized or logically represented. The hardware processor element 402 can also be configured or programmed to cause other devices to perform one or more operations as discussed above. In other words, the hardware processor element 402 may serve the function of a central controller directing other devices to perform the one or more operations as discussed above.

It should be noted that the present disclosure can be implemented in software and/or in a combination of software and hardware, e.g., using application specific integrated circuits (ASIC), a programmable logic array (PLA), including a field-programmable gate array (FPGA), or a state machine deployed on a hardware device, a computing device, or any other hardware equivalents, e.g., computer-readable instructions pertaining to the method(s) discussed above can be used to configure one or more hardware processor elements to perform the steps, functions and/or operations of the above disclosed method(s). In one example, instructions and data for the present module 405 for generating an instruction for a user to engage in a reduced performance level in response to detecting that a biometric output level of the user for a first physical activity via a first exercise apparatus is elevated as compared to a baseline biometric output level at a first performance level (e.g., a software program comprising computer-executable instructions) can be loaded into memory 404 and executed by hardware processor element 402 to implement the steps, functions or operations as discussed above in connection with the example method(s). Furthermore, when a hardware processor element executes instructions to perform operations, this could include the hardware processor element performing the operations directly and/or facilitating, directing, or cooperating with one or more additional hardware devices or components (e.g., a co-processor and the like) to perform the operations.

The processor (e.g., hardware processor element 402) executing the computer-readable instructions relating to the above described method(s) can be perceived as a programmed processor or a specialized processor. As such, the present module 405 for generating an instruction for a user to engage in a reduced performance level in response to detecting that a biometric output level of the user for a first physical activity via a first exercise apparatus is elevated as compared to a baseline biometric output level at a first performance level (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. Furthermore, a “tangible” computer-readable storage device or medium may comprise a physical device, a hardware device, or a device that is discernible by the touch. More specifically, the computer-readable storage device or medium may comprise any physical devices that provide the ability to store information such as instructions and/or data to be accessed by a processor or a computing device such as a computer or an application server.

While various examples have been described above, it should be understood that they have been presented by way of example only, and not limitation. Thus, the breadth and scope of a preferred example should not be limited by any of the above-described examples, but should be defined only in accordance with the following claims and their equivalents.

What is claimed is:

1. A method comprising:
obtaining, by a processing system including at least one processor, first biometric data of a user from at least one biometric device for a physical activity via at least a first exercise apparatus for a plurality of performance levels, wherein the obtaining includes associating biometric output levels from the first biometric data with corresponding performance levels of the plurality of performance levels as baseline biometric output levels;
detecting, by the processing system, a performance of the physical activity by the user via the at least the first exercise apparatus;
obtaining, by the processing system, a first performance level and second biometric data of the user associated with the performance of the physical activity, wherein the second biometric data indicates a first biometric output level;
detecting, by the processing system, that the first biometric output level of the user is an elevated level for the user at the first performance level; and
generating, by the processing system, an instruction for the user to engage in a reduced performance level for at least one of: the performance of the physical activity via the first exercise apparatus, a performance of at least a second physical activity via the first exercise apparatus, or the performance of the at least the second physical activity via a second exercise apparatus, wherein the reduced performance level is a lesser performance level as compared to the first performance level.

2. The method of claim 1, further comprising:
directing the user to engage in the first biometric output level for the performance of the physical activity via the first exercise apparatus.

3. The method of claim 1, further comprising:
directing the user to engage in the first performance level for the performance of the physical activity via the first exercise apparatus.

4. The method of claim 3, wherein the directing the user to engage in the first performance level comprises selecting at least a first setting of the first exercise apparatus, wherein the at least the first setting comprises at least one of:
a first resistance level;
a first weight load;
a first distance displacement;
a first maximal angle;
a first incline level; or
a first speed.

5. The method of claim 4, wherein the generating the instruction for the user to engage in the reduced performance level comprises selecting at least a second setting of the first exercise apparatus, wherein the at least the second setting comprises at least one of:
a second resistance level that is less than the first resistance level;
a second weight load that is less than the first weight load;
a second distance displacement that is less than the first distance displacement;
a second maximal angle that is less than the first maximal angle;
a second incline level that is less than the first incline level; or
a second speed that is less than the first speed.

6. The method of claim 1, wherein the first biometric data and the second biometric data both comprise at least one of:
pulse data;
breathing rate data;
pupil dilation data;
perspiration rate data;
blood oxygen saturation level data; or
temperature data.

7. The method of claim 1, wherein the plurality of performance levels comprises:
levels of speed;
levels of distance;
levels of distance displacement;
levels of angular contraction;
levels of time of performance;
levels of repetitions per weight; or
levels of repetitions per resistance.

8. The method of claim 1, wherein the generating the instruction for the user to engage in the reduced performance level for the performance of the at least the second physical activity via the first exercise apparatus comprises mapping the first performance level for the physical activity to a corresponding first performance level for the at least the second physical activity and wherein the reduced performance level is a lesser performance level as compared to the first performance level for the at least the second physical activity.

9. The method of claim 1, wherein the first physical activity comprises a first type of a resistance exercise or a weightlifting exercise and wherein the at least the second physical activity comprises at least a second type of resistance exercise or a second type of weightlifting exercise.

10. The method of claim 1, wherein the instruction for the user to engage in the reduced performance level is for the performance of the at least the second physical activity via the second exercise apparatus, the method further comprising:
transmitting the instruction to the second exercise apparatus to provide the reduced performance level via at least one setting of the second exercise apparatus.

11. The method of claim 10, wherein the second exercise apparatus is of a different exercise apparatus type as compared to the first exercise apparatus.

12. The method of claim 1, further comprising:
transmitting the instruction to an endpoint device of the user.

13. The method of claim 1, further comprising:
transmitting the instruction to the first exercise apparatus to provide the reduced performance level via at least one setting of the first exercise apparatus.

14. The method of claim 1, wherein the processing system is a processing system of an endpoint device of the user, wherein the method further comprises:
presenting the instruction as at least one of: an audio output or a visual output via the endpoint device.

15. The method of claim 1, further comprising:
presenting, in response to the detecting that the first biometric output level of the user is at the elevated level for the user at the first performance level, a query to the user as to whether the user is affected by a physiological limitation; and
obtaining an indication from the user that the user is affected by the physiological limitation, wherein the reduced performance level is selected for the user in response to the obtaining of the indication from the user that the user is affected by the physiological limitation.

16. A non-transitory computer-readable medium storing instructions which, when executed by a processing system including at least one processor, cause the processing system to perform operations, the operations comprising:
obtaining first biometric data of a user from at least one biometric device for a physical activity via at least a first exercise apparatus for a plurality of performance levels, wherein the obtaining includes associating biometric output levels from the first biometric data with corresponding performance levels of the plurality of performance levels as baseline biometric output levels;
detecting a performance of the physical activity by the user via the at least the first exercise apparatus;
obtaining a first performance level and second biometric data of the user associated with the performance of the physical activity, wherein the second biometric data indicates a first biometric output level;
detecting that the first biometric output level of the user is an elevated level for the user at the first performance level; and
generating an instruction for the user to engage in a reduced performance level for at least one of: the performance of the physical activity via the first exercise apparatus, a performance of at least a second physical activity via the first exercise apparatus, or the performance of the at least the second physical activity via a second exercise apparatus, wherein the reduced performance level is a lesser performance level as compared to the first performance level.

17. The non-transitory computer-readable medium of claim 16, the operations further comprising:
directing the user to engage in the first biometric output level for the performance of the physical activity via the first exercise apparatus.

18. The non-transitory computer-readable medium of claim 16, the operations further comprising:
directing the user to engage in the first performance level for the performance of the physical activity via the first exercise apparatus.

19. The non-transitory computer-readable medium of claim 18, wherein the directing the user to engage in the first performance level comprises selecting at least a first setting of the first exercise apparatus, wherein the at least the first setting comprises at least one of:
a first resistance level;
a first weight load;
a first incline level; or
a first speed.

20. An apparatus comprising:
a processing system including at least one processor; and
a computer-readable medium storing instructions which, when executed by the processing system, cause the processing system to perform operations, the operations comprising:
obtaining first biometric data of a user from at least one biometric device for a physical activity via at least a first exercise apparatus for a plurality of performance levels, wherein the obtaining includes associating biometric output levels from the first biometric data with corresponding performance levels of the plurality of performance levels as baseline biometric output levels;
detecting a performance of the physical activity by the user via the at least the first exercise apparatus;
obtaining a first performance level and second biometric data of the user associated with the performance of the physical activity, wherein the second biometric data indicates a first biometric output level;
detecting that the first biometric output level of the user is an elevated level for the user at the first performance level; and
generating an instruction for the user to engage in a reduced performance level for at least one of: the performance of the physical activity via the first exercise apparatus, a performance of at least a second physical activity via the first exercise apparatus, or the performance of the at least the second physical activity via a second exercise apparatus, wherein the reduced performance level is a lesser performance level as compared to the first performance level.
